(19) United States
(12) Patent Application Publication
(10) Pub. No.: US 2014/0171925 A1
(43) Pub. Date: Jun. 19, 2014

(54) APPARATUS FOR TREATING AN EYE USING A LASER BEAM

(71) Applicant: Taru Rummukainen, Vantaa (FI)

(72) Inventor: Taru Rummukainen, Vantaa (FI)

(73) Assignee: VALON LASERS OY, Vantaa (FI)

(21) Appl. No.: 14/008,520

(22) PCT Filed: Mar. 30, 2012
(86) PCT No.: PCT/FI2012/050319

§ 371 Date: Dec. 6, 2013

(30) Foreign Application Priority Data
Mar. 30, 2011 (FI) 20115302

(51) Int. Cl. A61F 9/008; A61B 3/00; A61B 3/14; A61B 3/135

(52) U.S. Cl. CPC A61F 9/00823; A61B 3/135; A61B 3/0008; A61B 3/0041; A61B 3/145
USPC 606/4

(57) ABSTRACT

An apparatus (1) for treating an eye using a laser beam includes a first light source (6) for producing a therapeutic laser beam (7), a second light source (8) for producing an alignment beam (9), and control means (2, 3, 4, 10) for controlling the first and the second light source and for directing the therapeutic laser beam and the alignment beam onto the retina (13) of the eye (12) being treated. According to the invention that the control means (2, 3, 4, 10) are arranged to control the second light source (8) and to direct the alignment beam (9) onto the retina (13) of the eye (12) being treated in such a way that the alignment beam forms a visual message (17) onto the retina as a function of at least one operational property of the therapeutic laser beam (7).

### FIELD OF THE INVENTION

The invention relates to apparatuses for treating an eye; specifically, to apparatuses for the purpose of laser treatment of the eye.

### BACKGROUND OF THE INVENTION

There are a number of diseases that may damage or deteriorate different parts of the human eye in different ways. A typical example of this is the damage caused by diabetes on the retina.

In some cases, the damaged sites of the eye can be treated by directing a laser beam of appropriate power thereto. A known example of such laser treatment is the treatment of the above-mentioned damages of the retina caused by diabetes by photocoagulation, i.e. burning by light. In photocoagulation, the energy of the laser beam is absorbed into the tissue of the retina, raising its temperature to above 65° C., whereupon the proteins are coagulated. As a result, the site of impact of the laser beam develops a dry scar, and progression of the damage is stopped.

The laser beam is directed and focused to the part of the eye being treated over the area of a small spot having a diameter typically ranging from a few dozen to some hundreds of micrometers. An area larger than this is treated by sequentially directing the laser beam to several adjacent spots, each exposed by the beam in a short pulse. Typically, the laser beam is directed sequentially to several adjacent spots, wherein they together constitute an area to be treated that is larger than a single spot. To move the laser beam to desired sites in the area being treated quickly and accurately, it is possible to use different for example galvanometric scanners. Known apparatuses have often preprogrammed different spot patterns comprising several spots of a therapeutic laser beam, among which a doctor or other user using the apparatus may select the most suitable one for each situation.

The exposure time for which the laser beam is directed to one spot may range for example from a few milliseconds to less than one second. The energy absorbed into a site being treated during the exposure time depends naturally on this exposure time and on the power of the therapeutic laser beam, typically ranging in known devices from about two hundred milliwatts to one or two watts.

For the purpose of directing the laser beam, the known apparatuses also have a second light source, for example a laser source having less power than the source producing the therapeutic laser beam, to form a so-called aiming or alignment beam. The apparatus is in this case arranged to steer the alignment beam to the same location in the area of the eye being treated as the therapeutic laser beam. The apparatus may be arranged for example to steer the therapeutic laser beam and the alignment beam to the eye through the same optics. The alignment beam of visible light in this case indicates the location of the therapeutic laser beam in the eye. In this way, the user of the apparatus may direct the apparatus to a desired site in the eye before activating the therapeutic laser beam. The alignment beam may be directed to the eye as one single beam. It can also be used to form a pattern according to a predetermined spot pattern of the therapeutic laser beam by directing the alignment beam in short sequential pulses to sites according to the spot pattern.

In dealing with treatment of an organ as sensitive as the eye, the apparatus being used in the treatment must naturally operate absolutely accurately and reliably. It is also essential that a doctor or other operator using the laser treatment apparatus is able to monitor the situation in the part of the eye being treated such as on the retina in real time, so as to be able to ensure appropriate directing of the laser beam and to monitor the effects provided by the laser beam on tissues. For this purpose, an imaging apparatus, for example a video camera and display or a combination of a slit lamp and a biomicroscope, is provided in the apparatuses for the laser treatment of the eye or in conjunction therewith, the imaging apparatus forming an image of the part of the eye being treated to be displayed to the user of the apparatus.

In addition to the location of the therapeutic laser beam and the optional spot pattern, it is essentially important for safety and success of the treatment that the user of the apparatus is constantly aware of operational properties of the therapeutic laser beam, such as power of the beam, exposure time and size of the spot. For example, power that is too low or too high or a wrong exposure time may lead to the energy absorbed into a site being treated that is either detrimentally high or too low for a therapeutic effect. The situation is complicated by the fact that, in most known apparatuses, at least one operational property of the therapeutic laser beam can be adjusted. For example, power of the beam may be selectable from several different alternatives.

In known apparatuses a typical user interface through which the user of the apparatus selects for example a spot pattern to be used or power of a therapeutic laser beam is presently based on a touch screen or a combination of a traditional screen and a separate input device such as a keyboard. The graphical user interface provided by such screens offers a clear and easy-to-use way to make choices in order to control operation of the apparatus. However, the known solutions have one significant problem. Even a graphical user interface displays data of currently selected operational settings of the apparatus physically separately from the actual object of the procedure, i.e. the eye being treated. In other words, in checking or adjusting the operational settings the user of the apparatus is forced in this case to look at the display device referred to. In this case, the person is forced to turn their eyes from the image formed of the eye being treated by means of a microscope or a video camera to the display device. This both stresses the user and slows down the executing of treatment procedures. The problem is particularly significant in the case of an imaging apparatus based on a microscope, where the user of the apparatus examines the area of the eye being treated through eyepiece of the microscope.

One example of the apparatuses according to the prior art is disclosed in publication U.S. Pat. No. 7,766,903 B2. Said publication discloses for example a principle of forming a selected spot pattern of a therapeutic laser beam onto the retina by means of an alignment beam.

### OBJECTIVE OF THE INVENTION

The objective of the invention is to disclose a novel apparatus for treatment of an eye by means of a laser beam, which alleviates the problems of the prior art referred to above.

### SUMMARY OF THE INVENTION

The invention is characterized by what has been presented in claim 1.

The invention relates to an apparatus for treating an eye using a laser beam, the apparatus including a first light source for producing a therapeutic laser beam, a second light source for producing an alignment beam and control means for controlling the first and the second light source and for directing the therapeutic laser beam and the alignment beam onto the retina of the eye being treated.

The first light source for producing the therapeutic laser beam may be of any known type applicable to the laser treatment of the eye. The type and properties of the light source producing the therapeutic laser beam are not essential for the basic principle of the invention. In the same way, this also applies to the second light source producing an alignment beam. As known from apparatuses on the market and from publications of the prior art, the second light source may be for example a visible wavelength low power laser source or an LED (light emitting diode) source. It is only essential that the second light source produces an alignment beam by means of which a site of impact of a therapeutic laser beam can be indicated on the retina of an eye being treated.

Control means refer herein to optics to be used in controlling the therapeutic laser beam and the alignment beam and in directing them to the eye, as well as to an electric control system controlling entire operation of the apparatus. The optics may comprise for example optical fibers or other photoconductors and for example a galvanometric scanner for quick and accurate locating and moving of the beams. The control means may also include one or more diffractive optical elements (DOE) for dividing the therapeutic laser beam into several subbeams. As the light sources, the control means may also be based on solutions and means known per se.

According to the invention, the control means are arranged to control the second light source and to direct the alignment beam onto the retina of an eye being treated in such a way that the alignment beam forms a visual message onto the retina as a function of at least one operational property of a therapeutic laser beam, i.e. data content visible on the retina. In other words, the basic principle of the invention is that, in addition to the location of the therapeutic laser beam, the alignment beam is used to display information of the operational properties of the therapeutic laser beam. The second light source, the control means and the alignment beam thus constitute display means for displaying said visual message on the retina of an eye being treated.

The message as a function of an operational property means in connection with this invention, as is clear even on a contextual basis, a message or data, the content of which is determined by state or value of a particular operational property in such a way that a user seeing the message may detect this state or value based on the message. “As a function of” in connection with this invention is thus not a mathematical definition, but instead represents generally any predetermined dependency or correspondence between content of the message and the operational property expressed thereby. It is thus a visual message corresponding to or dependent on said operational property. Examples of the way of realizing this correspondence or dependency are discussed below.

The operational properties of the therapeutic laser beam refer to those properties of the therapeutic laser beam which are relevant for execution and progress of the treatment. The operational property does thus not mean herein for example shape or location of a spot pattern being formed by the therapeutic laser beam on the retina of an eye being treated or in any other part of the eye. Said operational properties include for example power of the laser beam, exposure time of the therapeutic laser beam on the retina and size of the laser beam on the retina, i.e. size of a spot.

The two first mentioned properties referred to above are essentially important for the energy being absorbed into the area of the spot in a site of the eye being treated. The size of the spot that together with the power of the beam determines the intensity of the beam has in turn effect on the extent of the area this energy is distributed to and thus affects for example the rising of temperature caused by the energy in the tissue being treated. In an apparatus where the wavelength of the therapeutic laser beam or type of the laser source can be chosen from many different alternatives, it is possible, naturally, that the operational message comprises data of such choices. The operational property may also encompass the data on whether the therapeutic laser beam is actively on or not. This is essential data especially in situations where the wavelength of the therapeutic laser beam falls out of the wavelength range of visible light, in which case it is difficult or impossible to be directly detected.

The invention offers excellent advantages in terms of usability of an apparatus designed for the laser treatment of an eye. When data that is essential for the treatment on any property of a therapeutic laser beam is displayed on the retina of the eye being treated, the user of the apparatus, for example a doctor, need not turn their eyes from the retina to any separate display devide in checking the settings of the therapeutic laser beam, but instead they may concentrate the entire time on examining the actual area of the eye being treated. This calms down the treatment situation and essentially speeds up execution of the treatment.

A visual message of an operational property of a therapeutic laser beam may be displayed for example by means of different graphic images or symbols which are marked onto the retina or other part of an eye by means of an alignment beam. Specific predetermined symbols are a good alternative when there are only a small number of possible alternatives for a value of the operational property. If any property, for example power of the beam, may have any value over a specific control range, the use of separate symbols is limited. More flexible and varied possibilities are thus offered by a preferred embodiment of the invention where said visual message comprises text. The text means herein letters and/or numbers according to any writing system. Brightness of the site of impact of an alignment beam on the retina that can be adjusted by power of the alignment beam may also be part of the visual message.

In forming the visual message onto the retina it is possible to use means known per se for being used to control the alignment beam. In known laser treatment apparatuses for an eye there generally is a scanner which is used for deflection, i.e. moving, of the laser and the alignment beam as desired in an area of the eye being treated. The alignment beam is projected to the area being treated generally in sequential pulses of a short exposure time. After a pulse directed to one site, the beam is deflected to another site. When all the intended sites have been exposed, the same series is repeated, restarting from the first site of impact. Thus, it is possible to form for example a selected spot pattern of a therapeutic laser beam onto the retina or other site of an eye being treated by sequential pulses. When exposure times of the pulses are sufficiently short and repetition frequency of the sequential pulses is sufficiently high, the person using the apparatus sees, due to slowness of the human eye, the sites of impact of an alignment beam thus formed on the retina or other site of the eye being treated with the spots continuously illuminated. Such a principle of controlling the alignment beam for displaying the spot pattern of a therapeutic laser beam on the retina is described for example in publication U.S. Pat. No. 7,766,903 B2. In the apparatus according to the invention, the visual message of an operational property of the laser beam may be formed in a manner that is similar per se by moving the alignment beam according to the desired pattern or text. On the other hand, it is also possible that the control means in the apparatus according to the invention may include for example one or more diffractive optical elements for modifying the alignment beam in the manner required by the visual message. One essential difference of the invention to known solutions is that at least one operational property of a therapeutic laser beam is displayed instead of or in addition to a spot pattern of the therapeutic laser beam. The alignment beam may be arranged to operate in pulses or continuously.

The advantages of the invention are emphasized in an embodiment of the invention wherein the apparatus includes adjustment means for adjusting said at least one operational property of a therapeutic laser beam. In other words, in this embodiment the operational property of the therapeutic laser beam, as a function of which, in other words depending on which or corresponding to which, a visual message is formed by an alignment beam, can be adjusted. When the operational property, for example power of the therapeutic laser beam or exposure time, may have many different values, it is particularly important for a doctor or other person using the apparatus to know the currently selected value in order to avoid errors. When such a selected state or value of the operational property of the therapeutic laser beam is displayed directly on the retina according to the invention, the user of the apparatus may concentrate on examining the retina for the entire treatment procedure without interruptions.

The adjustment means naturally include means controlling the laser source and the optics controlling the laser source, but also adjustment devices by which the user of the apparatus may make choices concerning the operational properties of a therapeutic laser beam. The adjustment devices may basically be means known per se for controlling an apparatus. They may include for example different keys or other selectors and a display presenting selected adjustment values. However, in the case as referred to where an operational property displayed by means of a visual message can be adjusted, the adjustment means of the apparatus preferably include, either in addition to or instead of the traditional adjustment means, an adjustment device comprising a body portion and an actuator supported to be movable relative to the body portion for adjusting said at least one operational property of a therapeutic laser beam by moving the actuator. The operating principle of an adjustment device based on the movement of the actuator enables one to make choices of the operational properties of the therapeutic laser without eye contact to the actual adjustment device. This is an extremely significant difference in comparison to the adjustment means of known laser treatment apparatuses based on touch screens or traditional buttons where the user makes choices by touching the touch screen at specific sites or by pressing the buttons at specific sites of the apparatus. Traditional examples of an adjustment device based on moving an actuator include for example different joystick type adjustment devices.

In the adjustment device based on the movement of the actuator in the apparatus according to the invention the actuator preferably comprises an adjustment wheel arranged to rotate about a rotation axis relative to the body portion. Such a so-called jog wheel or scroll wheel type actuator is a particularly clear and easy-to-use solution in a treatment device demanding accuracy. Making choices by rotating the adjustment wheel does not require great concentration, in which case the user of the apparatus may uninterruptedly focus their attention on the retina being treated.

The apparatus according to the invention as described above, with the sources producing an alignment beam and a therapeutic laser beam and the control means, may be a module that is independent per se to be provided as part of other equipment used in the laser treatment of an eye. This other equipment typically includes for example means for supporting the head of a patient being treated in order to provide the laser treatment of the eye and means for examining the eye being treated. The latter includes for example imaging means for forming an image of the retina of the eye being treated for examining the area of the retina being treated. The imaging means may also be part of the apparatus according to the invention. In one embodiment of the invention, such imaging means as included in the apparatus comprise a slit lamp for illuminating the retina of an eye being treated and a biomicroscope for supplying a magnification of a site of the eye being treated to the user of the apparatus.

The imaging means constituting part of the apparatus may, on the other hand, also include a video camera and a display for presenting an image formed by the video camera.

### DETAILED DESCRIPTION OF THE INVENTION

Below, the invention will be described in detail with reference to preferred embodiments of the invention as presented in the accompanying figures.

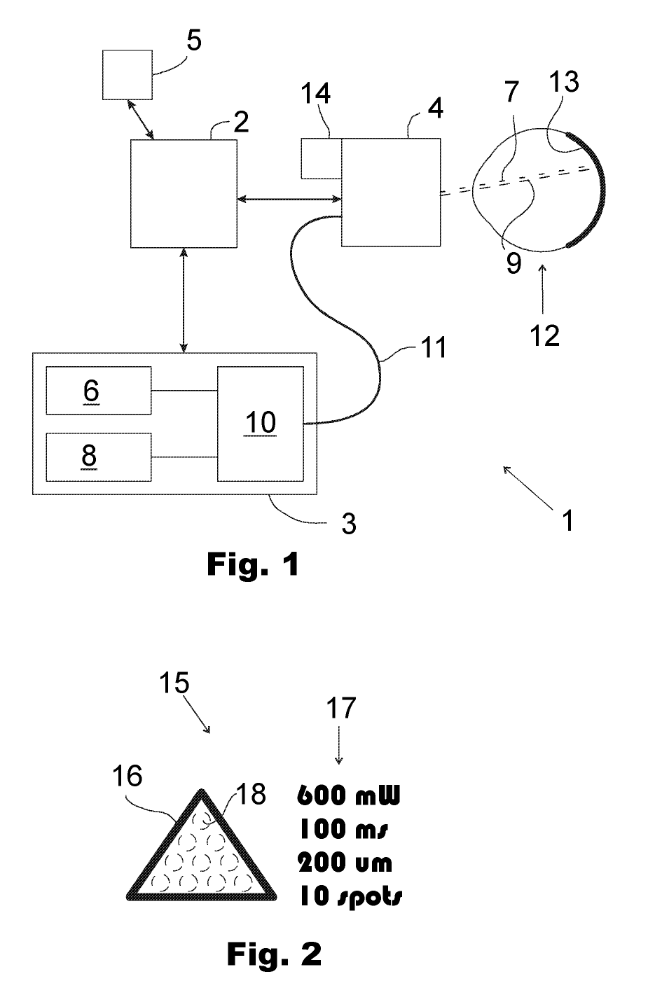

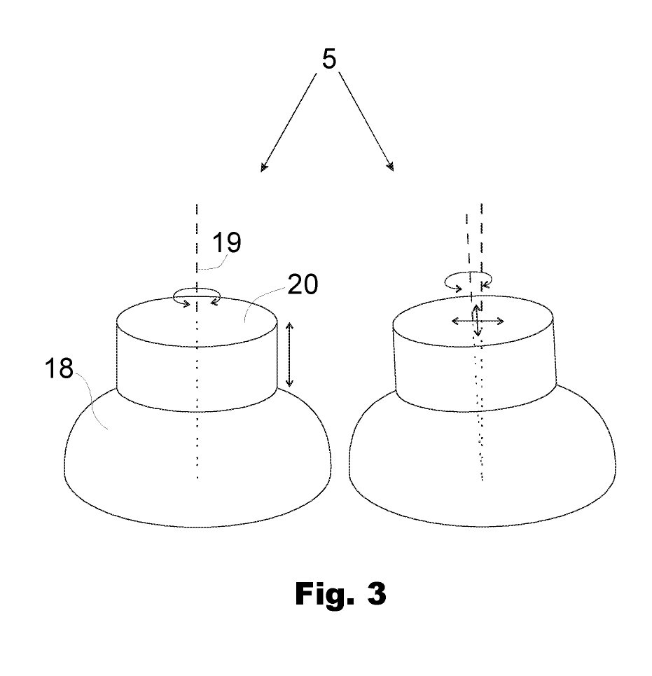

FIG. 1 is a schematic principle view of an apparatus for treating an eye by a laser beam. FIG. 2 illustrates properties and operation of the apparatus of FIG. 1. FIG. 3 shows an adjustment device as included in the apparatus according to FIG. 1.

The apparatus 1 of FIG. 1 includes a central processing unit 2, a source unit 3, an optics unit 4 and an adjustment device 5.

The source unit 3 comprises a laser source 6 for producing a therapeutic laser beam 7 and a second light source 8 for producing an alignment beam 9. The second light source 8 may be a low power laser source or for example an LED based source. The source unit 3 also comprises a coupling unit 10 connected to the sources 6, 8, with means for coupling the outputs of the laser source 6 and the second light source 8 one at a time to an optical cable 11 connecting the coupling unit 3 and the optics unit 4 to each other.

The optics unit 4 comprises means for modifying, controlling and directing the output of the laser source 6 or the second light source 8 that is currently introduced to the optics unit through the optical cable as a therapeutic laser beam 7 or an alignment beam 9, respectively, onto the retina 13 of an eye 12 being treated. These means may include different lenses, mirrors and other optical elements. For the purpose of accurate locating and moving of beams, the means may include for example a galvanometer based scanner.

The electric central processing unit 2 is operationally connected to the source unit 3 and the optics unit 4 to control them. The central processing unit 3 may include for example a computer and other electronics and software for controlling different means of the light sources, the coupling unit and the optics unit. It may also include a display device for forming graphical user interface of the apparatus 1.

The adjustment device 5 is operationally connected to the central processing unit 2 in such a way that the user of the apparatus 1 may control the apparatus by the adjustment device. By the adjustment device 5, the user may steer an alignment beam 9 and a therapeutic laser beam 7 to a desired location on the retina and make adjustments and choices concerning operation of the apparatus, in particular operational properties of the therapeutic laser beam 7.

In connection with the optics unit there are imaging means 14 which may include for example a slit lamp and a biomicroskope and/or a video camera, an image of which may be displayed for example by a display device of the central processing unit 2. The purpose of the imaging means 14 is to form a magnified image of the retina 13 of an eye being treated, from which image a person using the apparatus 1 may examine an area subject to treatment on the retina. The imaging means may partially consist of the optics of the optics unit 4 by using same optical components and systems of the optics unit in the apparatus for controlling an alignment beam 9 and a therapeutic laser beam 7 and to collect and steer light from the eye to the imaging means to form said image of the retina 13.

The apparatus of FIG. 1 and its parts may be based on components, devices and operating principles known per se, and any features relating to the above-said per se are not essential for the basic principle of the invention. In difference to that presented in FIG. 1, separate units may be integrated to one another or the apparatus may be divided in different units in a manner that is different from FIG. 1, and the scheme of FIG. 1 shall not be comprehended as limiting the invention in terms of assembly of the apparatus in any other respects.

The operating principle of the apparatus of FIG. 1 will be described below.

The user of the apparatus controls the central processing unit by the adjustment device 5 and the central processing unit 2 in turn controls the rest of the apparatus. Before actual treatment, the central processing unit of a therapeutic laser beam 7 controls the source unit 3 and its sources 6, 8 in such a way as to connect the optics unit 4 to the output of the light source 8. The central processing unit controls the optics unit in such a way as to direct an alignment beam 9 onto the retina 13 as a narrow cone. The light forming the alignment beam 9 travels in the optics unit 4 through the same optics as the light of the actual therapeutic laser beam 7, whereupon a site of impact of the alignment beam on the retina 13 corresponds to a location of a spot of the therapeutic laser beam or indicates an area of a predetermined spot pattern.

The apparatus 1 may be arranged to direct an alignment beam 9 onto the retina 13 in short, closely sequential pulses. When a site of impact of such beam on the retina is deflected to different sites of impact again and again in a specific path by means of the optics unit, the user of the apparatus sees the sites of impact along such path as continuously illuminated, provided that duration of the pulses is sufficiently short and repetition frequency and deflection rate of the beam are sufficiently high. Thus, by means of the alignment beam it is possible to form for example a selected spot pattern of a therapeutic laser beam onto the retina. On the other hand, it is essential in the invention that by suitably selecting the duration of pulses, repetition frequency and deflection rate of the beam, it is also possible to use the alignment beam to draw patterns seen by the user as continuous lines on the retina. Thus, in addition to a single spot or spot pattern, it is also possible to form text onto the retina.

In the apparatus of FIG. 1 the central processing unit 2, the source unit 3 and the optics unit 4 are arranged to control the second light source 8 and the entire apparatus 1 in such a way as to direct an alignment beam 9 onto the retina 13 of an eye 12 being treated in such a way that the alignment beam forms a visual message on the retina 13 as a function of at least one operational property of a therapeutic laser beam—i.e. depending on a state or value of the operational property as to its content. FIG. 2 presents one possible example on the apparatus arranged to operate in this way.

FIG. 2 shows a pattern 15 formed by the apparatus 1 of FIG. 1 onto the retina 13 of an eye 12 being treated by means of an alignment beam 9. The pattern 15 is formed by a frame 16 surrounding an area corresponding to the selected spot pattern of a therapeutic laser beam 7 and by an information field 17 drawn adjacent to this frame. The frame 16 and texts of the information field 17 are formed in a manner described above in such a way that the human eye sees them as being formed by solid lines. The following data is marked into the information field, one item below the other: power of a therapeutic laser beam, exposure time of a pulse of the therapeutic laser beam on the retina, diameter of the therapeutic laser beam on the retina and number of spots 18 of the selected spot pattern within the area delimited by the frame 16. By means of the data displayed in the information field, a doctor or other user using the apparatus sees the most essential operational properties of the therapeutic laser beam directly on the retina and thus need not remove their eyes from the image of the retina. They may also change one or some of said operational properties by means of the adjustment device 5 while for example looking the entire time at the image of the retina through a biomicroscope as included in the imaging means 14. Displaying essential operational data of a therapeutic laser directly on the retina makes using the apparatus 1 quick. It also improves safety and accuracy of the apparatus, as the selected settings of the therapeutic laser visible on the retina reduce possibility of directing the therapeutic laser beam to the eye with incorrectly selected operational properties.

When the optics unit 4 has been directed to a desired site on the retina by means of an alignment beam 9, and operational properties of a therapeutic laser beam 7 have been selected as desired by the adjustment device, the user may activate the therapeutic laser beam using the same adjustment device or for example a separate operating switch (not shown in the figure). In this case, the central processing unit 2 controls the source unit 3 and the optics unit 4 to steer the actual therapeutic laser beam 7 onto the retina instead of the alignment beam 9. The therapeutic laser beam 7 can be directed to a selected location on the retina in either one spot exposed in one or more sequential pulses, or, according to the example of FIG. 2, to sites of adjacent spots 18 according to a predetermined spot pattern in sequential pulses.

The adjustment device 5 of FIG. 3 is based on an adjustment wheel 20 supported to rotate about a rotation axis 19 relative to its base 18. The rotating adjustment wheel 20 is an especially well operating actuator for use without eye contact to the adjustment device 5. By rotational movement the user may give accurate control signals merely based on the rotational position of the adjustment wheel and its variance. In addition to rotational movement, the adjustment wheel 20 may also be for example tilted relative to the rotation axis 19, which is illustrated in the right-side drawing of FIG. 3, and pressed and/or lifted in the direction of this axis relative to the base 18 according to the vertical arrow indicated in the figure. The adjustment device 5 may also include separate buttons (not shown in the figure). Such additional directions of movement and/or additional buttons significantly increase the degrees of freedom of the adjustment device and diversify the control operations being provided by the adjustment device. The adjustment device according to the drawings of FIG. 3 may be a wireless control device or a traditional one connected to the rest of the apparatus 1 by a cable.

It is essential to notice that the invention is not limited to the examples described above, but that the embodiments of the invention may be freely varied within the scope of the claims.

1. An apparatus (1) for treating an eye using a laser beam, the apparatus including:
a first light source (6) for producing a therapeutic laser beam (7),
a second light source (8) for producing an alignment beam (9), and
control system (2, 3, 4, 10) for controlling the first and the second light source and for directing the therapeutic laser beam and the alignment beam onto the retina (13) of an eye (12) being treated,
characterized in that the control system (2, 3, 4, 10) is arranged to control the second light source (8) and to direct the alignment beam (9) onto the retina (13) of the eye (12) being treated in such a way that the alignment beam forms a visual message (17) onto the retina as a function of at least one operational property of the therapeutic laser beam (7).

2. The apparatus (1) according to claim 1, wherein said at least one operational property of the therapeutic laser beam (7) includes power of the therapeutic laser beam.

3. The apparatus (1) according to claim 1, wherein said at least one operational property of the therapeutic laser beam (7) includes exposure time of the therapeutic laser beam on the retina.

4. The apparatus (1) according to of claim 1, wherein said at least one operational property of the therapeutic laser beam (7) includes size of the therapeutic laser beam on the retina (13) of an eye (12) being treated.

5. The apparatus (1) according to claim 1, wherein said visual message (15) comprises text.

6. The apparatus (1) according to claim 1, including adjustment devices (2, 5) for adjusting said at least one operational property of the therapeutic laser beam (7).

7. The apparatus (1) according to claim 6, wherein the adjustment devices include an adjustment device (5) which comprises a body portion (18) and an actuator (20) supported to be movable relative to the body portion for adjusting said at least one operational property of the therapeutic laser beam (7) by moving the actuator.

8. The apparatus (1) according to claim 7, wherein the actuator comprises an adjustment wheel (20) arranged to rotate about a rotation axis (19) relative to the body portion.

9. The apparatus (1) according to claim 1, further comprising an imaging system (14) for forming an image of the retina (13) of an eye (12) being treated for examining the area of the retina being treated.

10. The apparatus (1) according to claim 9, wherein the imaging system (14) includes a slit lamp and a bio-microscope.

11. The apparatus (1) according to claim 9, wherein the imaging system (14) includes a video camera and a display for presenting an image formed by the video camera.
